(19) United States
(12) Patent Application Publication
(10) Pub. No.: US 2023/0383316 A1
(43) Pub. Date: Nov. 30, 2023

(54) BIOLOGICAL SINTERING OF CARBONATES WITHOUT HEAT OR PRESSURE

(71) Applicant: Biomason Inc., Durham, NC (US)

(72) Inventors: John Michael Dosier, Raleigh, NC (US); Ginger Krieg Dosier, Raleigh, NC (US)

(21) Appl. No.: 18/149,854

(22) Filed: Jan. 4, 2023

Related U.S. Application Data
(63) Continuation of application No. PCT/US2021/040537, filed on Jul. 6, 2021.
(60) Provisional application No. 63/048,844, filed on Jul. 7, 2020.

(51) Int. Cl. C12P 3/00; C01F 11/18; C04B 12/00; C04B 28/00

(52) U.S. Cl. CPC C12P 3/00; C01F 11/181; C04B 12/00; C04B 28/00

(57) ABSTRACT

Disclosed herein are compositions, tools and methods for the manufacture of construction materials, masonry, solid structures and compositions to facilitate dust control. More particularly, the disclosure is directed to the manufacture of bricks, masonry and other solid structures using small amount of aggregate material that is pre-loaded with spores and/or vegetative bacterial cells.

### CROSS-REFERENCE

This application is a continuation application of International Application No. PCT/US2021/040537, filed Jul. 6, 2021, which claims the benefit of U.S. Provisional Patent Application No. 63/048,844 filed Jul. 7, 2020, each of which application is incorporated herein by reference in its entirety.

### BACKGROUND

Traditional brick and concrete construction is heavily reliant on burning natural resources such as coal and wood. This reliance may result in the consumption of massive amounts of energy resources and equally massive carbon dioxide emissions, thus a great dependency on limited energy sources.

### SUMMARY

An alternative to the traditional processes for brick and concrete construction involves a process known as microbial induced calcite precipitation (MICP). MICP comprises mixing urease and urea as a source of energy with an aggregate material such as, for example, sand. The enzyme catalyzes the production of ammonia and carbon dioxide, increasing the pH level of the composition. A second enzyme, carbonic anhydrase, facilitates the transition of carbon dioxide into a carbonate anion. The rise in pH forms a mineral “precipitate,” combining calcium cations with carbonate anions. Particles present in the mixture act as nucleation sites, attracting mineral ions from the calcium forming calcite crystals. The mineral growth fills gaps between the sand particles biocementing or bonding them together. In some cases, the particles contain gaps of at least 5 microns in width but can be larger or smaller as desired. The resulting material exhibits a composition and physical properties similar to naturally formed masonry, bricks or other solid structures. Hardness can be predetermined based at least on the structure of the initial components and the pore size desired.

Enzyme producing bacteria that are capable of dissolving calcium carbonate include Alphaproteobacteria, Betaprobacteria, Gammaprobactreia, Firmicutes, or Actinobacteria. Enzyme producing bacteria that are capable of biocementation include Sporosarcina ureae, Proteus vulgaris, Bacillus sphaericus, Myxococcus xanthus, Proteus mirabilis, or Helicobacter pylori, although proper concerns should be given to pathogenic strains. Combinations of any of these strains as well as functional variants, mutations and genetically modified stains may be used as well. Bacterial compositions contain nutrient media to maintain and/or allow the cells to flourish and proliferate. The various types of nutrient media for cells, and in particular, bacterial cells of the disclosure may include at least minimal media (or transport media) typically used for transport to maintain viability without propagation, and yeast extract, and molasses, typically used for growth and propagation.

This method for manufacturing construction materials through induced cementation exhibits low embodied energy, and can occur at ambient pressure, and in a wide range of temperatures. The ambient temperature and conditions as well as the content of available aggregate can determine whether pure enzyme, lyophilized enzyme, or live cells are utilized as the starting components. Generally, live cells are used in warmer temperatures where mild weather conditions exist, whereas pure enzymes can be advantageous at more extreme conditions of cold or heat. The introduction of a bioengineered building unit using sand aggregate and naturally induced cementation provides a natural alternative that may be locally produced and environmentally friendly. As little to no heating is necessary, the energy savings in both expenses and efficiency is enormous.

Another advantage of MICP is that the process can be utilized in both small and large scale, and also easily automated. The bulk content of the masonry manufacturing process of the disclosure can be most any material that is locally available including rocks, sand, gravel and most any type of stone. Processing of the stone, such as crushing or breaking into pieces, also can be performed locally. Thus, transport costs and expenses are minimized. The composition of the disclosure (which may be provided lyophilized and hydrated on site), the frame for the bricks (if otherwise unavailable), and instructions as appropriate are all that need to be provided. If shipping is required, this represents a tiny fraction of the delivery costs, especially as compared to the present expenses associated with the delivery of conventional concrete.

Another advantage of the MICP process is to produce a “grown” construction material, such as a brick, utilizing primarily minerals, MICP and loose aggregate, such as sand. Not only can bricks and other construction materials be created, but the bricks themselves can be cemented into the desired places to “cement” bricks to one another and/or to other materials thereby forming the buildings, support structure or member, walls, roads, and other structures.

Biologically grown bricks and masonry do not require the traditional use of Portland cement mortar, which enables the reduction of atmospheric carbon dioxide by offering an alternative to the high-embodied energy traditionally manufactured construction materials. Employing cells to naturally induce mineral precipitation, combined with local aggregate and rapid manufacturing methods enables the production of a local, ecological, and economic building material for use throughout the global construction industry.

Although MICP can be utilized to create nearly any form of brick, block or solid structure used in construction, efficient methods for large scale manufacture have yet to be developed. Thus, a need exists for a rapid and convenient process that provides consistency to the manufacture of masonry that is both economical and environmentally safe. Also, the initial ingredients needed for MICP are not always readily available. Sources of calcium are often only available in the form of solid calcium carbonate. Thus, a need exists to obtain calcium.

The present disclosure overcomes problems and disadvantages associated with current strategies and designs, and provides new tools, compositions, and methods for the manufacture of building materials.

Disclosed herein are compositions, tools and methods of biological sintering involving the enzymatic break-down and reformation of calcium carbonate. In particular, the disclosure is directed to the manufacture of bricks, masonry and other solid structures, dust control, and the construction of roads, paths, and other solid surfaces using one or more enzymes that precipitate and/or dissolve calcium carbonate.

An aspect of the present disclosure provides a method comprising: (a) providing a first aqueous medium containing microorganisms which express enzymes that dissolve a carbonate mineral; (b) contacting the first aqueous medium with a carbonate mineral comprising magnesium carbonate (MgCO₃), ferric carbonate (FeCO₃), or dolomite (CaMg(CO₃)₂) under conditions that promote activity of the enzymes that dissolve the carbonate mineral to generate mineral ions and/or free carbon; and (c) collecting the mineral ions or free carbon.

In some embodiments, the first aqueous medium further comprises one or more of salts, amino acids, proteins, peptides, carbohydrates, saccharides, polysaccharides, fatty acids, oil, vitamins and minerals. In some embodiments, the microorganisms comprise one or more species, subspecies, strains, or serotypes of Alphaproteobacteria, Betaprobacteria, Gammaprobactreia, Firmicutes, or Actinobacteria. In some embodiments, the microorganisms comprise one or more species, subspecies, strains, or serotypes of Variovorax, Klebsiella, Pseudomonas, Bacillus, Exiguobacterium, Microbacterium, Curtobacterium, Rathayibacter, CellFimi2, Streptomyces, and/or Raoultella. In some embodiments, the method further comprises (d) providing a second aqueous medium containing microorganisms that express enzymes that form a carbonate mineral; and (e) contacting the second aqueous medium with the mineral ions or free carbon collected in (c) and a nitrogen source under conditions that promote activity of the enzymes contained in the second aqueous medium, to thereby produce a carbonate mineral using the mineral ions or free carbon. In some embodiments, the microorganisms contained in the second aqueous medium comprise one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori, and/or a urease and/or a carbonic anhydrase producing microorganism. In some embodiments, the contacting of (e) includes addition of a binding agent. In some embodiments, the binding agent comprises a polymer, a saccharide, a polysaccharide, a carbohydrate, a fatty acid, an oil, an amino acid, or a combination thereof. In some embodiments, the contacting of (b) and the contacting of (e) are performed substantially in parallel.

Another aspect of the present disclosure provides a method of manufacturing a material, the method comprising: (a) providing a first aqueous medium containing microorganisms which express enzymes that dissolve a carbonate mineral; (b) contacting the first aqueous medium with a carbonate mineral comprising magnesium carbonate (MgCO₃), ferric carbonate (FeCO₃), or dolomite (CaMg(CO₃)₂) under conditions that promote activity of the enzymes that dissolve the carbonate mineral to form calcium ions or free carbon; and (c) contacting the calcium ions or free carbon with a second aqueous medium containing microorganisms expressing enzymes that form a carbonate mineral using the calcium ions or free carbon, to thereby produce the material using the formed carbonate mineral.

In some embodiments, the material comprises a construction material. In some embodiments, the construction material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs. In some embodiments, the first or second aqueous medium comprises one or more of salts, amino acids, proteins, peptides, carbohydrates, saccharides, polysaccharides, fatty acids, oil, vitamins and minerals.

Another aspect of the present disclosure provides a method of manufacturing a construction material, the method comprising: (a) providing an aqueous medium that contains microorganisms which express a first enzyme that dissolves a carbonate mineral and microorganisms which express a second enzyme that form a carbonate mineral; and (b) contacting the aqueous medium with a carbonate mineral comprising magnesium carbonate (MgCO₃), ferric carbonate (FeCO₃), or dolomite (CaMg(CO₃)₂) under conditions that promote activities of both the first enzyme and the second enzyme to dissolve the carbonate mineral into mineral ions or free carbon, and form a carbonate mineral using the calcium ions or free carbon, to thereby manufacture the construction material.

In some embodiments, the construction material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs.

Another aspect of the present disclosure provides a composition comprising a first microorganism which expresses a first enzyme that dissolves a carbonate mineral, and a second microorganism which expresses a second enzyme that forms a carbonate mineral, the carbonate mineral comprising magnesium carbonate (MgCO₃), ferric carbonate (FeCO₃), or dolomite (CaMg(CO₃)₂).

In some embodiments, the first microorganism comprises one or more species, subspecies, strains, or serotypes of Alphaproteobacteria, Betaprobacteria, Gammaprobactreia, Firmicutes, or Actinobacteria. In some embodiments, the second microorganism comprises one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori. In some embodiments, the first microorganism or the second microorganism comprises spores. In some embodiments, the composition further comprises an aggregate. In some embodiments, the aggregate comprises sand, manufactured sand, crushed stone, crushed concrete, crushed brick, limestone, a silicate material, or a combination thereof. In some embodiments, the first microorganism is from 1.0 percent to 50 percent, by weight, of the composition suspended in a medium that maintains viability and does not promote growth or proliferation of the microorganisms. In some embodiments, the second microorganism is from 1.0 percent to 40 percent, by weight, of the composition suspended in a medium that maintains viability and does not promote growth or proliferation of the microorganisms. In some embodiments, the aggregate is from 10 percent to 95 percent, by weight, of the composition. In some embodiments, the composition comprises less than 10 percent, by weight, of water. In some embodiments, the composition comprises less than 5 percent, by weight, of water. In some embodiments, the composition comprises less than 2 percent, by weight, of water. In some embodiments, the composition comprises components that promote the germination and/or growth of the first and/or second microorganisms. In some embodiments, the components comprise nutrients, sugars, polysaccharides, stabilizers, preservatives, buffers, and/or salts. In some embodiments, the first and second microorganisms remain viable for about 6 months or longer. In some embodiments, the first and second microorganisms remain viable for about 12 months or longer. In some embodiments, the first and second microorganisms remain viable for about 24 months or longer. In some embodiments, the first microorganism or the second microorganism comprises spores. In some embodiments, the composition further comprises a calcium mineral.

Other embodiments and advantages of the invention are set forth in part in the description, which follows, and in part, may be obvious from this description, or may be learned from the practice of the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The manufacture of masonry and other building materials using a process known as microbial induced calcite precipitation (MICP) has been extensively described in a number of United States patent (e.g., see U.S. Pat. Nos. 8,728,365; 8,951,786; 9,199,880; and 9,428,418; each of which is incorporated in its entirety by reference). In these processes, urease-producing cells or urease enzymes are mixed with aggregate and incubated with urea and a calcium source. Calcite bonds form between aggregate particles resulting in a solid structure. Although the process allows for the manufacture of building materials, manufacturing generally requires standardization for the purpose of large-scale production.

It has been surprisingly discovered that the calcium can be collected from the dissolution of calcium carbonate by microorganisms which produce enzymes that dissolve calcium carbonate, and/or the enzymes themselves, thereby forming calcium ions and carbon ions. Microorganisms that produce enzymes that dissolve calcium carbonate include species, subspecies, strains, or serotypes of Alphaproteobacteria, Betaprobacteria, Gammaprobactreia, Firmicutes, or Actinobacteria such as, for example, species, subspecies, strains, or serotypes of Variovorax, Klebsiella, Pseudomonas, Bacillus, Exiguobacterium, Microbacterium, Curtobacterium, Rathayibacter, CellFimi2, Streptomyces, and/or Raoultella. The calcium ions produced by these enzymes and potentially the free carbon ions can be utilized by microorganisms that express enzymes that produce calcium carbonate. Microorganisms that produce enzymes that produce calcium carbonate include species, subspecies, strains or serotypes Sporosarcina pasteurii, Sporosarcina ureae, Proteus vulgaris, Bacillus sphaericus, Myxococcus xanthus, Proteus mirabilis, Bacillus megaterium, Helicobacter pylori, and/or any urease and/or carbonic anhydrase producing microorganism.

The process of biological sintering without heat or pressure utilizes microorganisms that produce enzymes that break down calcium carbonate as a calcium source that can be utilized for reformation of calcium carbonate using microorganisms that produce enzymes that form calcium carbonate. In a similar fashion, the dissolution of calcium also liberates carbon which can be used as the carbon source for calcium carbonate formation.

Calcium and calcium carbonate manufactured by enzymes can be standardized and, accordingly the manufacturing process enhanced. Standardization is achieved by adding an aqueous medium to a collection of viable bacteria forming an aqueous mixture and incubating the aqueous mixture under conditions that promote propagation. For cells that dissolve calcium carbonate, cells are mixed with calcium carbonate solids. For forming calcium carbonate, the cells or enzymes are mixed with the raw materials for forming calcium carbonate. Vegetative cells or enzymes can be mixed with particles (e.g., calcium carbonate particles or aggregate particles consistent with and/or similar to solid structure to be formed), forming a slurry and the slurry concentrated by the removal of at least a portion of the aqueous component, essentially the water, but not cells. Retention of cells can be achieved by utilizing aggregate particles of a size or average size and a composition that permits the transference of liquid such as water but retains cells. These ultrafine aggregate particles can be maintained as a slurry or further liquid can be removed as desired to form a powder or solid structure.

As disclosed herein including each of the various embodiments, although calcium carbonate (CaCO₃) is provided as an example material, other forms of carbonate may also be used. Other forms include but are not limited to magnesium carbonate (MgCO₃), ferric carbonate (FeCO₃), dolomite (CaMg(CO₃)₂), any form of carbonate (e.g., those minerals containing a carbonate ion {CO₃²⁻}), and any combination thereof. For both carbonate formation and dissolution, one can capture any carbonate mineral for dissolution and/or formation utilizing the enzymatic processes disclosed herein. These processes produce a hardened material or a compound ingredient production of a hardened material.

One embodiment of the disclosure is directed to a method for forming starter cultures of calcium carbonate dissolving and/or calcium carbonate forming microorganisms. Water and dissolved aqueous materials can be added or removed and the microorganisms as desired. Microorganism can be maintained as a slurry or dried as a powder or solid form. In some cases, the microorganisms are maintained in an aqueous or dried form that is relative resistant to variations in temperature or most any other external conditions, and therefore can be maintained for long periods of time. In this way, large numbers of microorganisms can be maintained to coordinate large manufacturing operations.

In a first step, spore-forming bacteria are cultured, e.g., under conditions that promote spore and/or vegetative cell formation. Culture conditions include an aqueous medium comprising one or more of salts, amino acids, proteins, peptides, carbohydrates, saccharides, polysaccharides, fatty acids, oil, vitamins and minerals. Non-limiting examples of calcium carbonate dissolving microorganisms comprise Variovorax, Klebsiella, Pseudomonas, Bacillus, Exiguobacterium, Microbacterium, Curtobacterium, Rathayibacter, CellFimi2, Streptomyces, and/or Raoultella. Non-limiting examples of calcium carbonate forming microorganism comprise one or more strains of Sporosarcina pasteurii, Sporosarcina ureae, Proteus vulgaris, Bacillus sphaericus, Myxococcus xanthus, Proteus mirabilis, Bacillus megaterium, Helicobacter pylori, and/or any urease and/or carbonic anhydrase producing microorganism. Microorganisms are maintained in minimal medium until use, and cultured in the aqueous medium, e.g., at incubation is at a physiological pH and at temperatures of from about 25-40° C. In some cases, incubation is performed from about 6 hours to about 6 days. In some cases, incubation is performed for about 1-3 days, or as short a time as necessary to generate the desired number of spores and/or vegetative cells per bacterium.

In some cases, spore formation or vegetative cell formation is induced, although an induction step is not required, and the microorganisms may be centrifuged or otherwise concentrated, and resuspended into a paste with media or another suitable liquid that maintains the microorganisms without inducing further growth and/or proliferation (a status solution). Alternatively, microorganisms may be mixed with aggregate without concentration, which may be for manufacturing batches of vegetative cells.

Also provided in the present disclosure is a composition for manufacturing a material which may be a building material or a construction material. Non-limiting examples of the material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs (e.g., concrete). The composition may comprise a mixture of microorganisms. The mixture of microorganism may comprise at least a first microorganism that dissolves a carbonate and a second microorganism that forms a carbonate. The first microorganism may express an enzyme that facilitates the dissolution of a carbonate. The second microorganism may express an enzyme that facilitates the formation of a carbonate. As described above or elsewhere herein, the carbonate may take any forms, e.g., a carbonate mineral comprising calcium carbonate (CaCO₃), magnesium carbonate (MgCO₃), ferric carbonate (FeCO₃), dolomite (CaMg(CO₃)₂), or any combination or variant thereof.

In some cases, the first microorganism or the second microorganism may comprise a group of microorganisms. In some cases, the dissolution and formation of the carbonate may occur under the same, substantially the same, or different conditions. Non-limiting examples of the first microorganism comprises one or more species, subspecies, strains, or serotypes of Alphaproteobacteria, Betaprobacteria, Gammaprobactreia, Firmicutes, Actinobacteria, or a combination thereof. Non-limiting examples of the second microorganism comprises one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori, or a combination thereof.

In some cases, the composition further contains an aggregate material, such as, for example, limestone, sand, a silicate material, or a combination thereof. The composition may comprise the aggregate material at a concentration from about 10 percent to about 99 percent, by weight (e.g., about 20 percent, about 30 percent, about 40 percent, about 50 percent, about 60 percent, about 70 percent about 80 percent, about 90 percent, about 95 percent), of the composition. Higher percentages of the aggregate material may be ready for subsequent uses whereas lower percentages of aggregate material may be then prepared in a concentrated form for storage or transport. In some cases, the first and second microorganisms combined constitute from about 10 percent to about 70 percent, by weight or higher (e.g., about 15 percent, about 20 percent, about 25 percent, about 30 percent, about 35 percent, about 40 percent, about 45 percent, about 50 percent, about 55 percent, about 60 percent, about 65 percent), of the composition. Higher percentages of the non-aggregate components of the composition can be for storage or transport use whereas lower percentages of the non-aggregate components may be ready for direct use. In some cases, the composition may not contain aggregate materials, and the aggregate materials may be added before use as desired for a given application. In some cases, the first and second microorganisms are present in relatively equal amount. However, in applications wherein there is a large amount of carbonate (e.g.,) calcium carbonate to be degraded, the first microorganism may predominate and, conversely, when there is a large quantity of carbonate (e.g., calcium carbonate) to be formed, the second microorganism may predominate. The amounts of each can be determined and adjusted as needed for a particular use. In some cases, the composition contains about 25 percent or less, by weight, of water, 20 percent or less, by weight, of water, 10 percent or less, by weight, of water, about 5 percent or less, by weight, of water, or about 2 percent or less, by weight, of water. The composition may also include components that support the germination and/or growth of the first and/or second microorganisms such as, for example, nutrients, sugars, polysaccharides, buffers, salts, stabilizers, preservatives.

In some cases, following spore-formation or vegetative cell formation as desired, cultures are mixed with aggregate particles. Aggregate particles may comprise natural, non-natural, recycled or manufactured sand, ore, crushed rock or stone, minerals, crushed or fractured glass, mine tailings, paper, waste materials, waste from a manufacturing process, plastics, polymers, roughened materials, and/or combinations thereof, and may be in the form of beads, grains, strands, fibers, flakes, crystals, or combinations thereof. The aggregate particles may comprise particles with a mesh size of 100 or smaller (particles of about 150 μm or smaller), 200 or smaller (particles of about 75 μm or smaller), or 300 or smaller (particles of about 38 μm or smaller).

The aqueous mixture of spores and/or vegetative cells and/or the aggregate is combined with a binding agent that promotes the adhesion or retention of microorganisms and aggregate. Adhesion may be between microorganisms and aggregate via hydrophobic bonds, hydrophilic bonds, ionic bonds, non-ionic bonds, covalent bonds, van der Waal forces, or a combination thereof. Binding agents may include, but are not limited to one or more of polymers, saccharides, polysaccharides, carbohydrates, peptides, proteins, fatty acids, oils, amino acids, or combinations thereof. In some cases, the binding agents are nontoxic and/or biodegradable and also harmless to the spores and do not interfere or otherwise hinder eventual germination of spores or proliferation of vegetative cells. Also, the composition may contain no toxins, toxic substances, or ingredients that pose a risk to the viability of the microorganisms or to individuals working with the composition or the final product.

In some cases, the aqueous component and mixture is removed is by evaporation and/or filtration, such as, for example, heat-assisted evaporation, pressure-assisted filtration, and/or vacuum-assisted filtration. Following evaporation and/or filtration, the slurry or aggregate particles and microorganisms contains from about 10⁶ to about 10¹⁴, from about 10⁸ to about 10¹², or from about 10⁹ to about 10¹¹ spores and/or cells/ml. The aqueous component can be further removed or removed entirely without hard to the spores and/or vegetative cells and the dried powder or block stored for future use in starting a culture of urease-producing bacteria.

Spore-containing aggregate material has a long shelf life. For example, shelf life produces greater than about 80 percent, about 90 percent, about 95 percent, or about 99 percent viability after about 3, about 6, about 9 or about 12 months of storage, or greater than about 80 percent, about 90 percent, about 95 percent, or about 99 percent viability after about 1, about 2, about 3 about 4, or about 5 years of storage. Vegetative-containing aggregate may have a shorter shelf life with greater than about 80 percent, about 90 percent, about 95 percent, or about 99 percent viability after about 1, about 2, about 3 about 4, about 5, or about 6 months of storage.

Another embodiment of the disclosure is directed to a composition comprising spore-loaded aggregate made by the methods of the disclosure. In some cases, aggregate particles are of a mesh size of 100 or smaller (particles of about 150 μm or smaller), 200 or smaller (particles of about 75 μm or smaller), or 300 or smaller (particles of about 38 μm or smaller). In some cases, the composition contains a binding or retention agent. The binding agent promotes adhesion between spores and/or vegetative cells and aggregate particles and/or the retention agent increases the size of aggregate particles and/or spores and/or vegetative cells, which promotes their retention.

In some cases, the composition contains less than about 50 percent liquid by weight, less than about 10 percent liquid by weight, or less than about 5 percent liquid by weight. In some cases, a composition may contain from about 10¹⁰ to about 10¹⁵ spores and/or vegetative cells/ml.

Another embodiment of the disclosure is directed to methods of manufacturing construction material comprising combining the dissolution of calcium carbonate with microorganisms and/or enzymes, followed by utilization of the calcium and/or carbon obtained from dissolution in the manufacture of calcium carbonate with microorganisms and/or enzymes. Solid calcium carbonate can be formed in a formwork or extruded as desired. Extruded calcium carbonate retains a basic shape upon extrusion that solidifies over time into a solid structure at a desired hardness.

The following examples illustrate embodiments of the disclosure and should not be viewed as limiting the scope of the disclosure.

### Example 1 Microorganism Production for Dissolution of Calcium Carbonate

Cultures of Variovorax, Klebsiella, Pseudomonas, Bacillus, Exiguobacterium, Microbacterium, Curtobacterium, Rathayibacter, CellFimi2, Streptomyces, and Raoultella. were produced from natural sources and from established cultures obtained from the American Type Culture Collection (ATCC). Cultures are maintained in minimal medium such as a pH balanced, salt solution to maintain viability without promoting proliferation or germination until ready for use.

### Example 2 Dissolution of Calcium Carbonate

Microorganisms of Example 1 are mixed with solid forms of calcium carbonate forming a slurry to which is added ingredients for growth and proliferation (e.g., which may include sugars, saccharides, polysaccharides, carbohydrates, fatty acids, lipids, vitamins, proteins, peptides, amino acids, salts, pH buffers, minerals, and/or additional components) as desired for the particular culture. The microorganisms dissolve the calcium carbonate and form calcium ions and free carbon.

### Example 3 Microorganism Production for Dissolution of Calcium Carbonate

Cultures of Sporosarcina pasteurii, Sporosarcina ureae, Proteus vulgaris, Bacillus sphaericus, Myxococcus xanthus, Proteus mirabilis, Bacillus megaterium, Helicobacter pylori were produced from natural sources and from established cultures obtained from the American Type Culture Collection (ATCC). Cultures are maintained in a minimal medium such as a pH balanced, salt solution to maintain viability without promoting proliferation or germination until ready for use.

### Example 4 Formation of Calcium Carbonate

Microorganisms of Example 3 are mixed with calcium ions and free carbon produced in accordance with Example 2 to which is added ingredients for growth and proliferation (e.g., which may include sugars, saccharides, polysaccharides, carbohydrates, fatty acids, lipids, vitamins, proteins, peptides, amino acids, minerals, salts, pH buffers and/or additional components) as desired for the particular culture. The microorganisms form calcium carbonate.

Other embodiments and uses of the disclosure will be apparent to those skilled in the art from consideration of the specification and practice of the disclosure disclosed herein. All references cited herein, including all publications, U.S. and foreign patents and patent applications, are specifically and entirely incorporated by reference. The term comprising, wherever used, is intended to include the terms consisting and consisting essentially of. Furthermore, the terms comprising, including, and containing are not intended to be limiting. It is intended that the specification and examples be considered exemplary only with the true scope and spirit of the disclosure indicated by the following claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-13. (canceled)

14. A method of manufacturing a construction material, the method comprising:
(a) providing an aqueous medium that contains microorganisms which express a first enzyme that dissolves a carbonate mineral and microorganisms which express a second enzyme that form a carbonate mineral; and
(b) contacting the aqueous medium with a carbonate mineral comprising magnesium carbonate (MgCO₃), ferric carbonate (FeCO₃), or dolomite (CaMg(CO₃)₂) and particles of an aggregate material under conditions that promote activities of both the first enzyme and the second enzyme to dissolve the carbonate mineral into mineral ions or free carbon, and
(c) forming a carbonate mineral to bind the particles together using the calcium ions or free carbon, to thereby manufacture the construction material.

15. The method of claim 14, wherein the construction material comprises bricks, thin bricks, pavers, panels, tile, veneer, cinder, breeze, besser, clinker or aerated blocks, counter- or table-tops, design structures, blocks, a solid masonry structure, piers, foundations, beams, walls, or slabs.

16. A composition comprising a first microorganism which expresses a first enzyme that dissolves a carbonate mineral, and a second microorganism which expresses a second enzyme that forms a carbonate mineral, the carbonate mineral comprising magnesium carbonate (MgCO₃), ferric carbonate (FeCO₃), or dolomite (CaMg(CO₃)₂) wherein the first microorganism comprises one or more species, subspecies, strains, or serotypes of Alphaproteobacteria, Betaprobacteria, Gammaprobactreia, Firmicutes, or Actinobacteria and the second microorganism comprises one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori.

17. (canceled)

18. (canceled)

19. The composition of claim 16, wherein the first microorganism or the second microorganism comprises spores.

20. The composition of claim 16, further comprising an aggregate.

21. The composition of claim 20, wherein the aggregate comprises sand, manufactured sand, crushed stone, crushed concrete, crushed brick, limestone, a silicate material, or a combination thereof.

22. The composition of claim 21, wherein the first microorganism is from 1.0 percent to 50 percent, by weight, of the composition suspended in a medium that maintains viability and does not promote growth or proliferation of the microorganisms and the second microorganism is from 1.0 percent to 40 percent, by weight, of the composition suspended in a medium that maintains viability and does not promote growth or proliferation of the microorganisms.

23. (canceled)

24. The composition of claim 21, wherein the aggregate is from 10 percent to 95 percent, by weight, of the composition and comprises less than 10 percent, by weight, of water.

25.-28. (canceled)

29. The composition of claim 16, comprising nutrients, sugars, polysaccharides, stabilizers, preservatives, buffers, and/or salts.

30. The composition of claim 16, wherein the first and second microorganisms remain viable for about 6 months or longer.

31. The composition of claim 30, wherein the first and second microorganisms remain viable for about 12 months or longer.

32. The composition of claim 31, wherein the first and second microorganisms remain viable for about 24 months or longer.

33. The composition of claim 32, wherein the first microorganism or the second microorganism comprises spores.

34. The composition of claim 33, further comprising a calcium mineral.

35. The method of claim 14, wherein the aqueous medium further comprises one or more of salts, amino acids, proteins, peptides, carbohydrates, saccharides, polysaccharides, fatty acids, oil, vitamins and minerals.

36. The method of claim 14, wherein the microorganisms comprise one or more species, subspecies, strains, or serotypes of Alphaproteobacteria, Betaprobacteria, Gammaprobacteria, Firmicutes, or Actinobacteria.

37. The method of claim 14, wherein the microorganisms comprise one or more species, subspecies, strains, or serotypes of Variovorax, Klebsiella, Pseudomonas, Bacillus, Exiguobacterium, Microbacterium, Curtobacterium, Rathayibacter, CellFimi2, Streptomyces, and/or Raoultella.

38. The method of claim 14, wherein the microorganisms comprise one or more species, subspecies, strains, or serotypes of Sporosarcina pasteurii, Sporosarcina ureae, Proteus vulgaris, Bacillus sphaericus, Myxococcus xanthus, Proteus mirabilis, Bacillus megaterium, Helicobacter pylori, and/or a urease and/or a carbonic anhydrase producing microorganism.

39. The method of claim 14, wherein the contacting of (b) includes addition of a binding agent.

40. The method of claim 39, wherein the binding agent comprises a polymer, a saccharide, a polysaccharide, a carbohydrate, a fatty acid, an oil, an amino acid, or a combination thereof.
